SEQUENCE LISTING (13 sequences)

SEQ ID NO: 1 - DNA, 300 bp, Rat
  misc_feature (1)..(300): angiotensin gene
tcacaaaaaacccattattccaggacacacagaagcaagtccacagatccgtgatgactcccacgggggcaggcctgaaggccaccatcttctgcatcctgacctgggtcagcctgacagctggggaccgcgtatacatccacccctttcatctcctctactacagcaagagcacctgcgcccagctggagaaccccagtgtggagacgctcccagagccaacctttgagcctgtgcccattcaggccaagacctcccccgtggatgagaagaccctgcgagataagctcgtgctgccca

SEQ ID NO: 2 - DNA, 24 bp, Artificial Sequence
  forward PCR primer
agcaagtccacagatccgtgatga

SEQ ID NO: 3 - DNA, 23 bp, Artificial Sequence
  reverse PCR primer
tgaatgggcacaggctcaaaggt

SEQ ID NO: 4 - DNA, 24 bp, Artificial Sequence
  forward PCR primer
atgactcccacgggggcaggcctg

SEQ ID NO: 5 - DNA, 27 bp, Artificial Sequence
  reverse PCR primer
ctaggggtggatgtatacgcggtcccc

SEQ ID NO: 6 - DNA, 23 bp, Artificial Sequence
  oligo linker containing a PacI site
aattgtcttaattaaccgcttaa

SEQ ID NO: 7 - DNA, 19 bp, Artificial Sequence
  oligonucleotide
aattgtcttaattaaccgc

SEQ ID NO: 8 - DNA, 19 bp, Artificial Sequence
  oligonucleotide
aattgcggttaattaagac

SEQ ID NO: 9 - DNA, 47 bp, Artificial Sequence
  primer LTR-1
ctgtacgtaccagtgcactggcctaggcatggaaaaatacataactg

SEQ ID NO: 10 - DNA, 63 bp, Artificial Sequence
  primer LTR-2
gcggatccttcgaaccatggtaagcttggtaccgctagcgttaaccgggcgactcagtcaatc

SEQ ID NO: 11 - DNA, 28 bp, Artificial Sequence
  primer HSA1
gcgccaccatgggcagagcgatggtggc

SEQ ID NO: 12 - DNA, 50 bp, Artificial Sequence
  primer HSA2
gttagatctaagcttgtcgacatcgatctactaacagtagagatgtagaa

SEQ ID NO: 13 - DNA, 10 bp, Artificial Sequence
  oligonucleotide
ttaagtcgac

(19) United States
(12) Patent Application Publication
(10) Pub. No.: US 2003/0073237 A1
(43) Pub. Date: Apr. 17, 2003

(54) Gene therapy to promote angiogenesis and/or the treatment of heart failure

(76) Inventors: Antonius Jacobus M. Roks, Groningen (NL); Yigal-Martin Pinto, Groningen (NL); Robert Henk Henning, Loppersum (NL); Wiekert Hendrikus van Gilst, Haren (NL)

Correspondence Address: TRASK BRITT, P.O. BOX 2550, SALT LAKE CITY, UT, 84110, US

(21) Appl. No.: 10/189,809

(22) Filed: Jul. 3, 2002

Related U.S. Application Data
(63) Continuation of application No. PCT/NL01/00005, filed on Jan. 4, 2001.
(60) Provisional application No. 60/176,172, filed on Jan. 13, 2000.

(30) Foreign Application Priority Data
Jul. 1, 2000 (EP) 00200071.9

(51) Int. Cl. A61K 48/00; C12N 15/861; C12N 15/86

(52) U.S. Cl. 435/456; 424/93.2; 435/320.1

(57) ABSTRACT

Nucleic acid delivery vehicles for enhancing and/or inducing angiogenesis are provided. The delivery vehicles comprise a nucleic acid encoding angiotensin 1-7 or a functional part, derivative and/or analogue thereof. The vehicles are among others suited for the treatment of heart failure.

### CROSS-REFERENCE TO RELATED APPLICATION

This application is a continuation of co-pending International Application Number PCT/NL01/00005 filed on Jan. 4, 2001, designating the United States of America, International Publication No. WO 01/49325 (Jul. 12, 2001), the contents of the entirety of which is incorporated by this reference.

### TECHNICAL FIELD

The present invention relates to the field of human gene therapy, particularly to gene therapy vehicles for treating cardiovascular disease and methods and means to improve cardiac performance, for the treatment of heart failure.

### BACKGROUND

Hypertension and hypercholesterolemia are two of the main risk factors for human health in the Western world. These conditions can lead to atherosclerosis. Atherosclerosis may result in a number of severe cardiovascular diseases, like chronic heart failure, angina pectoris, claudicatio intermittens, or peripheral and myocardial ischemia. At least the early phases of atherosclerosis are characterized by endothelial dysfunction. Endothelial dysfunction causes coronary arterial constriction and plays a role in both hypertension and hypercholesterolemia. It is one of the first measurable steps in the cascade of reactions leading to atherosclerosis, even before macroscopic lesions are evident. Many therapies have been investigated to assess the possibility to reverse the endothelial dysfunction, and to stimulate the formation of new blood vessels (angiogenesis) Examples are cholesterol reduction and ACE-inhibition. It has been suggested that oral L-arginine supplementation in the diet may be a therapeutic strategy to improve angiogenesis in patients with endothelial dysfunction.

It is well established that angiogenesis is mediated by a multitude of cytokines (like TNF-α and E-selectin) and angiogenic factors including basic Fibroblast Growth Factor (bFGF), Vascular Endothelial Growth Factor (VEGF), and TGF-β. Both bFGF and VEGF are key regulators of angiogenesis in adult tissues. They selectively stimulate proliferation of endothelial cells, starting with the binding of these growth factors to receptors present on the endothelial cell surface. Nitric oxide (NO) has been shown to play a role in this process. NO, originally identified as endothelium-derived relaxing factor, is an important endothelial vasoactive factor.

While both NO and angiogenic factors like bFGF and VEGF play a key role in the endothelial functions, their precise mode of action is not known. On the one hand, levels of angiogenic factors like bFGF and VEGF are increased in patients suffering from endothelial dysfunction. On the other hand, the release of nitric oxide in vascular endothelial dysfunction is often reduced. This reduced release may cause constriction of the coronary arteries and thus contribute to heart disease. It is postulated that patients suffering from endothelial dysfunction could benefit from therapies to increase new collateral blood vessel formation and/or therapies to increase vasodilatation.

Many experimental gene therapies concentrate on the stimulation of angiogenesis, in patients suffering from endothelial dysfunction, through the addition of VEGF or bFGF. Though these experimental therapies may have some effect, the level of therapy-induced angiogenesis is low, leading to a slow, if at all, recovery or enhancement of blood flow. The induction of angiogenesis is considered to be particularly relevant for cardiac related diseases. While for most tissue other than the heart, reduced blood flow is severely debilitating, reduced blood flow in the heart muscle is life threatening.

Cardiac tissue contains roughly two compartments consisting of cardio-myocytes and non-myocytes, respectively. The cardiomyocytes are highly differentiated cells which have lost the ability to divide, and can adapt only by enlargement, so-called hypertrophy. The non-myocyte compartment consists of cells like fibroblasts, macrophages, vascular smooth muscle cells, vascular endothelial cells, endocardial cells and of an extracellular matrix. Enlargement of the non-myocyte compartment can be achieved by cell division and matrix deposition.

Physiological enlargement during normal development and growth, and in response to intense exercise is characterized by an equal increase in both compartments. As a result, total myocardial contractility is increased. In contrast, myocardial adaptation in response to pressure/volume overload or myocardial infarction characteristically disturbs normal myocardial architecture, resulting in a relative increase of extracellular matrix and a decrease in capillary density^{1,2}. The relative deficit of capillaries in turn is the trigger for development of ischemia, which leads to deterioration of cardiac function on the long-term.

The RAS (Renin Angiotensin System) is considered one of the most important regulatory systems for cardiovascular homeostasis. It plays a central role in blood pressure regulation, and in growth processes in the vessel wall as well as the myocardium^{3,4}. The key enzyme, the angiotensin converting enzyme (ACE), which is abundantly present on endothelial cells, activates Ang II and inactivates bradykinin (BK). Ang II, which is formed from Ang I by ACE, is a vasoconstrictor and growth stimulator when acting on the AT1 receptor while BK is a potent vasodilator. BK is degraded by ACE through sequential removal of the dipeptides Phe-Arg and Ser-Pro from the C-terminal end of the decapeptide. In addition to their inhibitory effect on Ang II formation, accumulation (and potentiation) of endogenous BK may be another mechanism by which ACE-inhibitors exert their effects⁵.

The beneficial effects of ACE-inhibitors on hypertrophied myocardium have been described extensively in animal and in human studies³. Treatment with ACE-inhibitors not only reduces symptoms, but also improves survival in heart failure patients⁴. Ang II is a potent growth factor for myocytes, fibroblasts, and vascular smooth muscle cell (VSMC). On a cellular level multiple mechanisms play a role. Next to oncogenes and cyclins⁶, interference with cell cycle regulating homeobox genes may be important. Ang II promotes unwanted VSMC proliferation by downregulation of cell cycle arresting genes such as the growth arrest homeobox (gax)⁷. In this context, it is interesting that gene transfer with gax reduces porcine in-stent restenosis⁸.

The effect of BK on cell proliferation is less well described. It has been suggested that BK reduces fibroblast and VSMC proliferation by a prostaglandin- and NO-dependent mechanism. Given all the above, therefore, it is not surprising that upregulation of (cardiac) ACE activity as found after myocardial infarction contributes to unfavorable remodeling of the myocardium: cardiomyocyte hypertrophy, increased matrix, and relative deficit of neovascularisation or angiogenesis.

Angiogenesis—sprouting of new capillaries from the pre-existing vascular network—rarely occurs in the heart under normal conditions. Ang II has been described as an angiogenic factor^{9,10} while at the same time ACE-inhibitors also have been described to exert angiogenesis promoting activity¹¹⁻¹⁴. Although this seems contradictory, it might be explained by the stimulating effect of Ang II on VSMC to produce and release VEGF (mediated by the AT₁ receptor) which is a potent angiogenic factor¹⁵.

As already mentioned, ACE inhibition interferes not only with Ang II formation but also with the breakdown of BK. Since BK stimulates angiogenesis through BK₁ receptors and Ang II inhibits angiogenesis through AT₂-receptor¹⁶ mediated inhibition of endothelial cell (EC) proliferation¹⁵, both effects of ACE inhibition may be pro-angiogenic in itself. Interference with the RAS may therefore have a dual synergistic effect. Reduction of hypertrophy and extracellular matrix formation on the one hand and stimulation of angiogenesis on the other hand.

### SUMMARY OF THE INVENTION

In the present invention, it was found that, RAS interference by Ang (1-7), a member of circulating angiotensin peptides, prevents heart failure, presumably due to a synergism between reducing specific growth processes like myocardial and vascular hypotrophy on the one hand and by stimulating myocardial angiogenesis on the other hand. It seems promising, therefore, to further identify specific components of the RAS with regard to these specific actions.

For this reason, we also have studied Ang II generating systems alternative to ACE such as chymase¹⁷⁻¹⁹. Biochemical and pharmacological studies suggest an important role for chymase in total Ang II capacity—especially in human tissues—although its exact relevance in (patho-) physiology remains uncertain.

The present invention makes use of the notion that heptapeptide Ang 1-7, a member of circulating angiotensin peptides, which levels seem to be increased after ACE-inhibition, functions as an endogenous inhibitor of the PAS. We show that Ang 1-7 antagonizes the vasoconstrictor effects of Ang I and II. It has been shown that Ang 1-7 enhances bradykinin B₂ receptor mediated vasodilatation, displays antihypertensive actions in rats and inhibits cultured rat VSMC growth. Importantly, since Ang(1-7) in addition causes cardiac NO release, application of Ang(1-7) in a gene therapy setting results in improved perfusion of the heart muscle, both directly through vasodilatation and indirectly through stimulation of NO-mediated angiogenesis. This is the first demonstration that Ang (1-7) or a functional derivative thereof is capable of preventing and/or inhibiting heart failure.

Animal and cell culture studies demonstrate that Ang 1-7 inhibits ACE activity, antagonizes AT₁ receptors, enhances BK-induced vasodilatation, and stimulates NO release via an Ang 1-7 receptor^{20-22,23-25}. This leads to the concept that Ang 1-7 is be an endogenous counterplayer of the renin-angiotensin system through a wide variety of mechanisms²⁶. The present invention employs the properties of Ang 1-7 to modulate local growth processes in order to restore the balance between above described compartments and normalize myocardial architecture, and to make comparisons to other known growth modulators such as NO and VEGF. For this purpose, newly developed gene transfer vectors are used to induce specific and localized overexpression of these modulator substances at the site of interest.

### BRIEF DESCRIPTION OF THE DRAWINGS

FIG. 1 is a schematic representation of the amplified genomic DNA regions and primer sets for PCR in production of a Ang-(1-7)-coding nucleotide sequence (SEQ ID NO:1).

### DETAILED DESCRIPTION OF THE INVENTION

In one aspect, the invention provides a delivery vehicle for at least in part preventing heart failure including a means for the release of angiotensin 1-7, preferably in the vicinity of the cardiac muscle. In a preferred aspect the delivery vehicle includes a nucleic acid delivery vehicle for enhancing and/or inducing angiogenesis including a novel nucleic acid including at least one sequence encoding angiotensin 1-7 or a functional part, derivative and/or analogue thereof, and further including a nucleic acid delivery carrier. For the present invention a functional analogue of angiotensin 1-7 is angiotensin 1-9/Ang(1-9) Since Ang(l-9) like Ang(1-7) is an ACE inhibitor (Kokonen et al. Circulation 1997, 95:1455-1463), and since both angiotensins re-sensitize the Bradykinin receptor (Marcic et al. Hypertension, 1999, 33, 835-843). A functional part, derivative and/or analogue of Ang(1-7) and/or Ang(1-9) includes the same cardiac hyperthrophy inhibiting and/or preventing activity combined with myocardial angiogenesis stimulating activity in kind not necessarily in amount.

When in the present invention angiotensin 1-7 is referred to, this reference includes a functional part, derivative and/or analogue of angiotensin 1-7. Angiotensin 1-7 is effective since it has an intrinsic vasodilatating effect in coronary arteries. Moreover, angiotensin 1-7 stimulates AT, receptor which is a counterregulator of the unfavorable AT, receptor. Furthermore, angiotensin 1-7 stimulates the giving of, of prostacycline which inhibits vasoconstriction. In a preferred embodiment, the nucleic acid delivery vehicle further includes at least one sequence encoding an additional angiogenesis promoting factor. These may suitable be chosen from the group of VEGF, bFGF, angiopoietin-1, a nucleic acid encoding a protein capable of promoting nitric oxide production, and functional analogues or derivatives thereof. Surprisingly, it has been found that, under certain circumstances, a synergistic effect is obtained in the enhancing and/or inducing angiogenic effect. The additional angiogenesis promoting factors may be supplied by sequences provided by the nucleic acid delivery vehicle or provided in other ways. They may also be provided by cells transduced or cells in the vicinity of surrounding transduced cells. In a preferred embodiment, the expression of at least one of the sequences is regulated by a signal. Preferably, the signal is provided by the oxygen tension in a cell. Preferably, the oxygen tension signal is translated into a different expression by a hypoxia inducible factor 1α promoter. Considering that RAS is activated in a number of cardiovascular afflictions, promoters of the gene coding for ACE and the genes coding for angiotensin receptors are also preferred. An advantage of such a promoter is that the transcription of an RAS-inhibitor (Angiotensin 1-7), is turned on upon activation of transcription of unfavorable RAS components. Such a mechanism enables a production of Angiotensin 1-7 predominantly when there is a need for it, thus obviating at least in part other control mechanisms for targeting expression to relevant cells.

In another aspect of the invention, the nucleic acid delivery vehicle may further include a sequence encoding a herpes simplex virus thymidine kinase, thus providing an additional method of regulating the level of enhanced and/or induced angiogenesis. The level may at least in part be reduced through the addition of gancyclovir, killing not only at least in part the dividing cells in the newly forming vessel parts, but also killing at least in part transduced cells thereby limiting the supply of nitric oxide and/or additionally angiogenesis promoting factors.

The nucleic acid delivery carrier may be any nucleic acid delivery carrier, such as a liposome or virus particle. In a preferred embodiment of the invention the nucleic acid delivery carrier includes a Semliki Forest virus (SFV) vector, an adenovirus vector or an adeno-associated virus vector preferably including at least essential parts of SFV DNA, adenovirus vector DNA and/or adeno-associated virus vector DNA. Preferably a nucleic acid delivery vehicle has been provided with at least a partial tissue tropism for muscle cells. Preferably a nucleic acid delivery vehicle has been at least in part deprived of a tissue tropism for liver cells. Preferably the tissue tropism is provided or deprived at least in part through a tissue tropism determining part of fiber protein of a subgroup B adenovirus. A preferred subgroup B adenovirus is adenovirus 16.

In another aspect, the invention provides a method for at least in part improving myocardial architecture including providing cells of an individual, preferably a mammal, more preferably a human, with a nucleic acid delivery vehicle according to the invention and culturing the cells, preferably in vivo, under conditions allowing expression of a protein capable of increasing nitric oxide production.

In another aspect, the invention provides a method for at least in part reducing hypertrophy including providing cells of an individual, preferably a mammal, more preferably a human, with a nucleic acid delivery vehicle according to the invention and culturing the cells, preferably in vivo, under conditions allowing expression of a protein capable of increasing nitric oxide production.

In another aspect, the invention provides a method for enhancing and/or inducing angiogenesis including providing cells of an individual, preferably a mammal, more preferably a human, with a nucleic acid delivery vehicle according to the invention and allowing the cells to be cultured under conditions allowing expression of a protein capable of increasing nitric oxide production. As has been mentioned herein, the method may be a method for enhancing and/or inducing angiogenesis in a synergistic fashion with at least one additional angiogenesis promoting factor or parts or derivatives or functional analogues thereof. Preferably the enhancing and/or inducing angiogenesis effect is at least in part reversible. Preferably, the effect is at least in part reversed though an increase in the oxygen tension or through providing the cells with gancyclovir or functional analogue thereof, or both.

In a preferred aspect of the invention, at least cells are transduced that under normal circumstances are not in direct contact with blood. The advantage being that in this way the treatment promotes at least in part the localization of the effect. Preferably the cells not in direct contact with the blood are muscle cells, preferably cardiac or skeletal muscle cells, more preferably smooth muscle cells. Highly preferred cells in this regard are located in the heart of an individual suffering from or at risk of suffering from heart pressure overload and/or myocardial infarction. When feasible, a preferred means of providing cells with a nucleic acid delivery vehicle of the invention is a catheter, preferably an Infiltrator catheter (see, e.g., European patent application EP 97200330.5). In another preferred method for providing cells with a nucleic acid delivery vehicle of the invention, the cells are provided with the nucleic acid delivery vehicle through pericardial delivery, preferably by a so-called perducer. Pericardial delivery is preferred since it limits the delivery to the relevant organ. Moreover, pericardial delivery is preferred since it results in a more even improvement of cardiac architecture.

In one aspect, the invention provides the use of a nucleic acid delivery vehicle or a method for the treatment of endothelial dysfunction. In one embodiment the use increases at least in part vasodilatation of constricted vessels. In another embodiment the use increases at least in part angiogenesis, be it enhanced or induced or both.

In another aspect, the invention provides a cell for the production of an adenovirus vector or an adeno-associated virus vector of the invention wherein the cell includes a means for the production of the virus vector in the absence of replication competent adenovirus and adeno-associated virus. In a preferred embodiment the cell expresses at least one means for the production of the virus vector from a nucleic acid integrated in the chromosomal DNA of the cell and expresses other means for the production of the virus vector from nucleic acid not integrated in the chromosomal DNA of the cell and wherein the integrated nucleic acid and the non-integrated nucleic acid, do not include sequence overlap leading to the formation of replication competent adenovirus. In a particularly preferred embodiment the integrated nucleic acid includes at least an adenovirus E1-region. In another particularly preferred embodiment the integrated nucleic acid includes at least a sequence encoding an adenovirus E2A protein, preferably an E2A-protein derived from adenovirus ts125. In another preferred embodiment the integrated nucleic acid includes an adenovirus E4-region, preferably E4-orf6. Preferably, the cell is derived from a PER.C6 cell (ECACC deposit number 96022940).

It is clear that when reference is made herein to a certain protein, that for the purpose of the invention also functional analogues or derivatives of the protein may be used, wherein the functional analogue possesses the same kind of activity though not necessarily the same amount of activity.

The invention will now be elucidated by the following, illustrative examples.

### EXAMPLES

### Example I

Methods for the expression of a Angiotensin-(1-7)-coding nucleotide sequence based on polymerase chain reaction.

Angiotensin-(1-7) (Ang-(1-7)) is a hormone of the renin-angiotensin system, and has effects that counteract Angiotensin II (Ang 11)²⁸. Overactivity of Aug II has been related to various cardiovascular diseases such as hypertension, atherosclerosis, restenosis after PTCA, and heart failure. Hence, Ang-(1-7) could be an important hormone that beneficially influences cardiovascular disease. Therefore, Ang-(1-7) producing vectors may be of clinical interest.

In the circulatory system, Ang-(1-7) is formed through metabolism of Ang I by neutral endopeptidases EC 3.4.21.26 (NEP 24.11)²⁷. In an attempt to artificially augment Ang-(1-7) expression, we produced recombinant Semliki Forest virus (SFV) that contains a nucleotide sequence coding for Ang-(1 -7) preceded by the pro-angiotensinogen signal peptide. The production method of the Ang-(1-7)-coding sequence is based on a simple two-step PCR technique using smooth muscle cell genomic DNA (gDNA).

PCR Primer Sets and Reaction Conditions.

A cDNA sequence coding for Ang-(1-7) preceded by the N-terminal signal peptide of angiotensinogen was produced by two consecutive PCR steps.

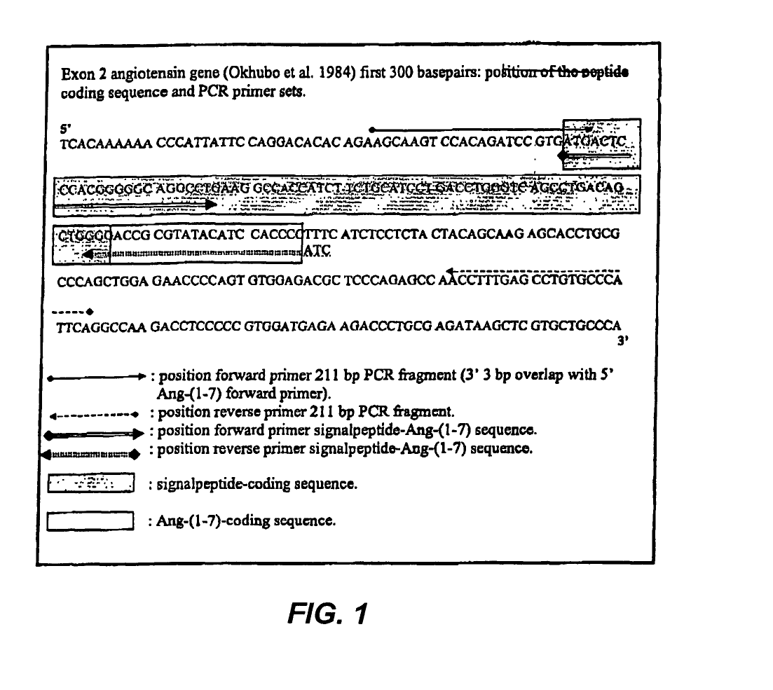

First PCR step: PCR primers were constructed to amplify 211 bp of exon 2 of the rat angiotensin gene (SEQ ID NO:1) published by Ohkubo et al. (1984) from rat genomic DNA (FIG. 1). Forward primer sequence: 5′-AGC AAG TCC ACA GAT CCG TGA TGA-3′ (SEQ ID NO:2). Reverse primer sequence: 5′-TGA ATG GGC ACA GGC TCA AAG GT-3′ (SEQ ID NO:3). A 50 μl PCR reaction mixture containing 0.5 units Taq polymerase (Eurogentec, Belgium), 5 ,μl of the supplied lO×PCR buffer, 17.5 nmol dNTP, 2 mM MgCl₂, 50 ng rat genomic DNA, 75 pg forward and 75 pg reverse primer. Temperature cycling was performed in 0.5 ml thin-walled vials in a thermal cycler (DNA thermal cycler, Perkin Elmar, USA) using 1 minute periods of 94° C. (DNA strand separation), 56° C. (primer annealing), and 72° C. (primer extension). A total of 20 cycles were run. The 211 bp PCR product was isolated by gel electrophoresis and subsequent extraction of the cDNA from the gel with the Qiagen extraction kit.

Second PCR step: The 211 bp PCR product was used for further PCR. A primer set designed to amplify a Ang-(1-7) coding sequence, consisting of the 72 bp pro-angiotensinogen signal peptide coding sequence followed by the 21 bp Ang-(1-7) coding sequence (FIG. 1), further denoted as SPAng-(1-7) Additionally, the 5′ end of the reverse primer consisted of the antisense triplet 5′-CTA-3′ to produce the sense stop codon 5-TAG-3′. Sequence forward primer: 5′-ATG ACT CCC ACG GGG GCA GGC CTG-3′ (SEQ ID NO:4). Sequence reverse primer: 5′-CTA GGG GTG GAT GTA TAC GCG GTC CCC-3′ (SEQ ID NO:5). The 96 bp PCR product was ligated into pGEM-T-Easy (Promega) to form pGEM-T:SPAng-(1-7) which was used for further processing.

### Example II

Generation of an SFV Vector Including Ang(1-7)

Ligation of the SPAng-(1-7) coding region in pSFV2.

After ligation, pGEM-T:SPAng-(1 -7) was transformed and amplified into E. ccli JM 109. LacZ negative colonies were used to amplify pGEM-T:SPAng-(1-7). The Ang-(1-7) coding sequence was cut out of pGEM-T-Easy with the restriction enzymes Nco I and Spe I. The single strand overhang was made blunt ended by a fill-in reaction with Klenow pclymerase. Then, the blunt ended SPAng-(1-7) coding sequence was ligated into pSFV2 linearized with Sma I to form pSFV2-SPAng-(1-7). The pSFV2-SPAng-(1-7) double stranded DNA plasmid was converted into single stranded RNA plasmid via the SP6 reverse transcriptase initiation site present in pSFV2. This RNA plasmid was used for production of SFV-Ang-(1-7) virus.

Production of SFV-Ang-(1-7) Virus.

SFV-helper 2 particles were produced and titers assessed in BHK 21 cells as described previously²⁸.

### Example III

Generation of an Adenovirus Vector Including Ang (1-7)

Generation of Specific Plasmids/Cosmids for the Generation of Adenovirus Vectors.

pBr/Ad.C1a-Bam (ECACC deposit P97082117)

wt Adeno type 5 DNA was digested with ClaI and BamHI, and the 20.6 kb fragment was isolated from gel by electro-elution. pBr322 was digested with the same enzymes and purified from agarose gel by Geneclean. Both fragments were ligated and transformed into competent DH5α. The resulting clone pBr/Ad.Cla-Bam was analyzed by restriction enzyme digestion and shown to contain an insert with adenovirus sequences from bp 919 to 21566.

pBr/Ad.AfII-Bam (ECACC deDosit P97082114)

Clone pBr/Ad.Cla-Bam was linearized with EcoRI (in pBr322) and partially digested with AflII. After heat inactivation of AflII for 20 minutes at 65° C., the fragment ends were filled in with Klenow enzyme. The DNA was then ligated to a blunt double stranded oligo linker containing a PacI site (5′-AATTGTCTTAATTAACCGCTTAA-3′) (SEQ ID NO:6). This linker was made by annealing the following two oligonucleotides: 5′-AATTGTCTTAATTAACCGC-3′ (SEQ ID NO:7) and 5′-AATTGCGGTTAATTAAGAC-3′ (SEQ ID NO:8), followed by blunting with Klenow enzyme. After precipitation of the ligated DNA to change buffer, the ligations were digested with an excess PacI enzyme to remove concatameres of the oligo. The 22016 bp partial fragment containing Ad5 sequences from bp 3534 up to 21566 and the vector sequences, was isolated in LMP agarose (SeaPlaque GTG), religated and transformed into competent DH5α. One clone that was found to contain the PacI site and that had retained the large adeno fragment was selected and sequenced at the 5′ end to verify correct insertion of the PacI linker in the (lost) AflII site.

pWE/Ad.AflII-rITR (ECACC Deposit P97082116)

Cosmid vector pWE15 (Clontech) was used to clone larger Ad5 inserts. First, a linker containing a unique PacI site was inserted in the EcoRI sites of pWE15 creating pWE.pac. To this end, the double stranded PacI oligo as described for pBr/Ad.AflII-BamHI was used but now with its EcoRI protruding ends. The following fragments were then isolated by electroelution from agarose gel:pWE.pac digested with PacI, pBr/AflII-Bam digested with PacI and BamHI and pBr/Ad.Bam-rITR#2 digested with BamHI and PacI. These fragments were listed together and packaged using λ. phage packaging extracts (Stratagene) according to the manufacturer's protocol. After infection into host bacteria, colonies were grown on plates and analyzed for presence of the complete insert.

pWE/Ad.AflII-rITR contains all adenovirus type 5 sequences, from bp 3534 (AflII site) up to and including the right ITR (missing the most 3′ G residue).

Construction of an Adapter Plasmid.

Generation of Adapter Plasmid pAd/L420-HSApac

The absence of sequence overlap between the recombinant adenovirus and E1 sequences in the packaging cell line is essential for safe, RCA-free generation and propagation of new recombinant viruses. The adapter plasmid pMLPI.TK (described in WO 97/00326) is an example of an adapter plasmid designed for use in combination with improved packaging cell lines like PER.C6 (described in WO 97/00326 and U.S. Ser. No. 08/892,873). This plasmid was used as the starting material to make new adapter plasmids in which nucleic acid molecules including specific promoter and gene sequences can be easily exchanged.

First, a PCR fragment was generated from pzipΔMo+PyF1O1 (N⁻) template DNA (described in PCT/NL96/00195) with the following primers: LTR-1: 5′-CTG TAC GTA CCA GTG CAC TGG CCT AGG CAT GGA AAA ATA CAT AAC TG-3′ (SEQ ID NO:9) and LTR-2: 5′-GCG GAT CCT TCG AAC CAT GGT AAG CTT GGT ACC GCT AGC GTT AAC CGG GCG ACT CAG TCA ATC G-3′ (SEQ ID NO:10). Pwo DNA polymerase (Boehringer Mannheim) was used according to manufacturers protocol with the following temperature cycles: once 5′ at 95° C.; 3′ at 55° C.; and 1′ at 72° C., and 30 cycles of 1′ at 95° C., 1′ at 60° C., 1′ at 72° C., followed by once 10′ at 72° C. The PCR product was then digested with BamHI and ligated into pMLP10 (Levrero et al., 1991) vector digested with PvuII and BamHI, thereby generating vector pLTR10. This vector contains adenoviral sequences from bp 1 up to bp 454 followed by a promoter consisting of a part of the Mo-MuLV LTR having its wild-type enhancer sequences replaced by the enhancer from a mutant polyoma virus (PyF101). The promoter fragment was designated L420. Next, the coding region of the murine HSA gene was inserted. pLTR10 was digested with BstBI followed by Klenow treatment and digestion with NcoI. The HSA gene was obtained by PCR amplification on pUC18-HSA (Kay et al., 1990) using the following primers: HSA1, 5′-GCG CCA CCA TGG GCA GAG CGA TGG TGG C-3′ (SEQ ID NO: 11) and HSA2, 5′-GTT AGA TCT AAG CTT GTC GAC ATC GAT CTA CTA ACA GTA GAG ATG TAG AA-3′ (SEQ ID NO:12). The 269 bp amplified fragment was subcloned in a shuttle vector using the NcoI and BglII sites. Sequencing confirmed incorporation of the correct coding sequence of the HSA gene, but with an extra TAG insertion directly following the TAG stop codon. The coding region of the HSA gene, including the TAG duplication was then excised as a NcoI(sticky)-SalI(blunt) fragment and cloned into the 3.5 kb NcoI(sticky)/BstBI(blunt) fragment from pLTR10, resulting in pLTR-HSA10.

Finally, pLTR-HSA10 was digested with EcoRI and BamHI after which the fragment containing the left ITR, packaging signal, L420 promoter and HSA gene was inserted into vector pMLPI.TK digested with the same enzymes and thereby replacing the promoter and gene sequences. This resulted in the new adapter plasmid pAd/L420-HSA that contains convenient recognition sites for various restriction enzymes around the promoter and gene sequences.

Generation of pAd5.CLIPsal.

Another adapter plasmid that was designed to allow easy exchange of nucleic acid molecules was made by replacing the promoter, gene and poly A sequences in pAd/L420-HSA with the CMV promoter, a multiple cloning site, an intron and a poly-A signal. For this purpose, pAd/L420-HSA was digested with AvrII and BglII followed by treatment with Klenow to obtain blunt ends. The 5.1 kb fragment with pBr322 vector and adenoviral sequences was isolated and ligated to a blunt 1570 bp fragment from pcDNA1/amp (Invitrogen) obtained by digestion with HhaI and AvrII followed by treatment with T4 DNA polymerase. This adapter plasmid was named pAdS/CLIP. To enable removal of vector sequences from the left ITR in pAd5/Clip, this plasmid was partially digested with EcoRI and the linear fragment was isolated. An oligo of the sequence 5′ TTAAGTCGAC-3′ (SEQ ID NO:13) was annealed to itself resulting in a linker with a SalI site and EcoRI overhang. The linker was ligated to the partially digested pAd5/Clip vector and clones were selected that had the linker inserted in the EcoRI site 23 bp upstream of the left adenovirus ITR in pAd5/Clip resulting in pAdS/Clipsal.

Construction of Recombinant Adenoviral Vector pAd5/CLIP.Ang1-7.

Plasmid pGEM-T:SPAng-(1-7) containing the Ang1-7 sequence was digested with SstII, and the 3′ protruding ends were filled in using T4 DNA polymerase. After purification of the DNA using Qiaquick nucleotide removal kit (Qiagen), the Ang1-7 fragment was excised from the plasmid backbone by digestion with NotI. In parallel the adapter plasmid pAd5/Clipsal was first digested with XbaI, blunted with klenow and, after purification, digested with NotI.

The 6.7 kb NotI-XbaI digested vector fragment from pAd5/Clipsal was separated from undigested material and linker sequences on a 1% LMP gel (Sea Plaque GTG agarose, FMC Bioproducts) in a 1×TAE buffer. The band with vector DNA was excised from the gel and dephosphorylated in the LMP agarose using Tsap enzyme (Gibco). The 126 bp Ang1-7 sequence was separated from the vector backbone on a 2% LMP gel and ligated to the dephosphorylated vector fragment. Following transformation into DH5alpha bacteria, clones were selected that contained the Ang1-7 insert.

Virus was then generated by co-transfection in PER.C6 cells of pAd5/Clip.Ang1-7 digested with SalI and pWE/Ad.AflII-rITR digested with PacI using 4 microgram of each DNA. Virus was plaque purified and propagated using standard methods for the plaque purification and propagation of E1-deleted adenovirus vectors and E1-expression cell lines.

All transfections were performed by calcium-phosphate precipitation DNA (Graham et al., (1973) Virology 52:456-467) with GIBCO Calcium Phosphate transfection System (GIBCO BRL life technologies, Inc., Gaithersburg, Md., USA), according to the manufacturer's protocol.

Measurement of Ang-(1-7) Production.

A7r5 rat smooth muscle cells were grown in 25 cm² culture flasks to confluent cell cultures. Cells were washed with PBS and 2 ml DMEM without FCS was added per flask. Then, 10⁸ lU/ml SFV-Ang-(1-7) or SFV3-LacZ was added per flask. For Ad-Ang-(1-7), 100 μl 4 Ad-Ang-(1-7) or Ad-luciferase virus stock was added per flask. A non-treated control group was included as well. The cells transfected with SFV or Ad and control cells were grown for 24 h (SFV-treated and control cells) or 48 h (Ad-treated cells). After this, cultured medium was removed and immediately stored at −80° C. To the cells, 500 μl demineralized water was added after which the cells were immediately frozen and stored for 20 h at −20° C. Then, the cells were thawed and scraped on ice, and the lysate thus obtained was stored at −80° C. until measurement of Ang-(1-7) Ang-(1-7) was measured with the use of a radio immuno assay (RIA).

A7r5 rat aortic smooth muscle cells were cultured in Dulbecco's Modified Eagles medium, containing 100 U/L streptomycin, 100 μg/L penicillin, 20 mmol/L HEPES, and 10% fetal calf serum (FCS)

Both SFV-Ang-(1-7) virus and Ad-Ang-(1-7) virus were transfected to A7r5 rat aortic smooth muscle cells. Levels of Ang-(1-7) were calculated as fmoles per total sample, see table 1. Transfection with either of the vectors resulted in increased levels of Ang-(1-7) in the media as well as in the cell lysates.

TABLE 1
(data Angl-7 production)

| Sample | RIA data (fmoles Ang(1-7)) |
|---|---|
| media Ad-angl-7 | 313 |
| control media | 87 |
| lysate Ad-angl-7 | 2056 |
| control lysate | 1036 |
| media SFV angl-7 | 874 |
| control media | 83 |
| lysate SFV angl-7 | 1394 |
| control lysate | 1036 |

### REFERENCES

1. Weber K T, Brilla 0 G. Pathological hypertrophy and cardiac interstitium. fibrosis and renin-angiotensin-aldosteron system. Circulation 1991;83:1849-1865.

2. Weber K T, Cardiac interstitium in health and disease: the fibrillar collagen network. J Am Coll Cardiol 1989;13:1637-1652.

3. Brown N J, Vaughan D E. Angiotensin-converting enzyme inhibitors Circulation 1998;97:1411-1420.

4. Chryssant S G. Vascular remodelling: The role of angiotensin converting enzyme inhibitors. Am Heart J 1998;135(Spt2):S21-S30.

5. Campbell D J, Kladis A, Duncan A-M. Effects of converting enzyme inhibitors on angiotensin and bradykinin peptides. Hypertension 1994;23:439-449.

6. Diez J, Panizo A, Hernandez M, Galindo M F, Cenarruzabeitir E, Parodo Mindan F J. Quinapril inhibits c-Myc expression and normalizes smooth muscle cell proliferation in spontaneous hypertensive rats. Am J Hypertens 1997;10(Pt2):1147-1152.

7. Yamashita-J; Itoh-H; Ogawa-Y; Tamura-N; Takaya-K; Igaki-T; Doi-K; Chun-T H; Inoue-M; Masatsugu-K; Nakao-K Opposite regulation of Gax homeobox expression by angiotensin II and C-type natriuretic peptide. Hypertension. 1997(1Pt2):381-387.

8. Tio R A, Smith R C, Scheuermann T H, Lebherz C, Luo Z, Tkebuchava T, Duverger N, Branellec D, Isner J M, Walsh K. Adenovirus mediated Gax gene therapy in stented porcine coronary arteries: delivery, dissemination and, efficacy. (submitted).

9. Amant C, Bauters C, Bodart J-C, Lablanche J-M, Grollier G, Danchin N, Hamon M, Richard F, Helbecque N, McFadden E, Amouyel P, Bertrand M. D Allele of the angiotensin I-converting enzyme is a major risk factor for restenosis after coronary stenting. Circulation 1997;96:56-60.

10. Fernandez L A, Twickler J, Mead A. Neovascularisatin produced by angiotensin II. J Lab Olin Med. 1985;105:141-145.

11. le Noble FAC, Schreurs N H J S, van Straaten H W M, Slaaf J F M, Rogg H, Struijker-Boudier A J. Evidence for a novel angiotensin II receptor involved in angiogenesis in chick chorioallantoic membrane. Am J Physiol 1993;264:R460-R465.

12. Olivetti G, Cigola B, Lagrasta C, Ricci R, Quaini F, Mnopoli A, Ongini E. Spirapril prevents left ventricular hypertrophy, decreases myocardial damage and promotes angiogenesis in spontaneously hypertensive rats. J Cardiovasc Pharmacol 1993;21:362-370.

13. Unger T, Mattfeldt T, Lamberty V, Bock P, Mall G, Linz W, Scholkens B W, Gohlke P. Effects of early onset angiotensin converting enzyme inhibition on myocardial capillaries. J Hypert 1992;20:487-482.

14. Cameron N E, Cotter M A, Robertson S. Angiogtensin converting enzyme inhibition prevents the development of the muscle and nerve dysfunction and stimulates the angiogenesis in streptozotocin-diabetic rats. Diabetologia 1992;35:12-18.

15. Stoll M, Meffert 5, Stroth U, Unger T. Growth or antigrowth: angiotensin and the endothelium. J Hypert 1995;13:1529-1534.

16. Hu D E, Fran T P D [Leu⁸]des-Arg⁹-bradykinin inhibits the angiogenic effects of bradykinin and interleukin-1 in rats. Br J Pharmacol 1993;109:14-17.

17. Voors A A, Pinto Y M, Buikema H, Utara H, Rooks G, Grandjean J G, D. Ganten, van Gilst W H. Dual pathway for angiotensin II formation in human internal mammary arteries. Br J Pharmacol (accepted).

18. Roks A J M, Buikema H, Pinto Y M, van Gilst W H. The renin-angiotensin system and vascular function. The role of angiotensin II, angiotensin-converting enzyme, and alternative conversion of angiotensin I. Heart Vessels 1997;S12:119-124.

19. Nishimura H, Buikema H, Baltatu O, Ganten D, Urata H. Functional evidence for alternative angiotensin II-forming pathways in hamster cardiovascular system Am J Physiol (accepted)

20. Paula R D. Lima C V, Khosla M C, Santos R A S. Angiotensin-(1-7) potentiates the hypotensive effects of bradykinin in conscious rats. Hypertension 1995;26:1154-1159.

21. Deddish P A, Marcic B, Jackman H L, Wang H-Z, Skidgel P A, Erdös E G. N-domain-specific substrate and C-domain inhibitors of angiotensin-converting enzyme. Angiotensin(1-7) and Keto-ACE. Hypertension 1998;31:912-917.

22. Brosnihan K B, Li P, Ferrario C M. Angiotensin-(1-7) dilates canine coronary arteries through kinins and nitric oxide. Hypertension 1996;27:523-528.

23. Mahon J M, Carr R D. Nicol A K, Henderson I W. Angiotensin-(1-7) is an antagonist at the type 1 angiotensin II receptor. J Hypertens 1994;12:1377-1381.

24. Pörsti I, Bara A, Busse R, Hecker M. Release of nitric oxide by angiotensin-(1-7) from porcine coronary endothelium: implications for a novel angiotensin receptor. Br J Pharmacol 1994;111:625-654.

25. Tallant E A, Lu X, Weiss R B, Chapell M C, Ferario C M. Bovine aortic endothelial cells contain an angiotensin-(1-7)receptor. Hypertension 1997;29(2):388-393.

26. Ferario C M, Chapell M C, Tallant A, Brosnihan B, Diz D I. Counter-regulatory actions of angiotensin-(1-7) Hypertension 1997;30(2):535-541.

27. Yamamoto K, Chappell M C, Brosnihan K B, Ferrario C M. In vivo metabolism of angiotensin I by neutral endopeptidase (EC 3.4.24.11) in spontaneously hypertensive rats. Hypertension. 1992;19:692-696.

28. Berglund P, Sjöberg M, Garoff H, Atkins G J, Sheahan B J, Liljestrom P. Semliki Forest virus expression system: production of conditionally infectious recombinant particles. Bio/technol. 1993;11:916-920.

What is claimed is:

1. A nucleic acid delivery vehicle for enhancing and/or inducing angiogenesis, said nucleic acid delivery vehicle comprising:
a nucleic acid comprising at least one sequence encoding an angiotensin 1-7 or a functional part, derivative and/or analogue thereof, and
a nucleic acid carrier.

2. The nucleic acid delivery vehicle of claim 1, further comprising at least one sequence encoding an additional angiogenesis promoting factor.

3. The nucleic acid delivery vehicle of claim 2, wherein said additional angiogenesis promoting factor is selected from the group consisting of VEGF, bFGF, angiopoietin-1, and a nucleic acid encoding a protein capable of promoting nitric oxide production.

4. The nucleic acid delivery vehicle of claim 1, claim 2, or claim 3, wherein the expression of at least one sequence is regulated by a signal.

5. The nucleic acid delivery vehicle of claim 4, wherein said signal is provided by oxygen tension.

6. The nucleic acid delivery vehicle of any of claim 1, claim 2, claim 3, claim 4, or claim 5 wherein said nucleic acid delivery carrier is selected from the group consisting of a liposome, a virus particle, and a functional analogue or derivative thereof.

7. The nucleic acid delivery vehicle of claim 6, wherein said nucleic acid delivery carrier comprises a Semliki Forest virus vector, an adenovirus vector or an adeno-associated virus vector.

8. A method for enhancing and/or inducing angiogenesis, said method comprising:
providing cells of a subject with the nucleic acid delivery vehicle of any of claims 1-7.

9. The method according to claim 8, wherein said enhancing and/or inducing angiogenesis effect is at least partly reversible.

10. The method according to claim 9, wherein said effect is at least in part reversed through an increase in the oxygen tension or through providing said cells with gancyclovir or a functional analogue thereof, or both.

11. The method according to any one of claims 8-10, wherein said cells comprise at least cells that under normal circumstances are not in direct contact with blood.

12. The method according to claim 11, wherein said cells are muscle cells.

13. The method according to claim 12, wherein said muscle cells are cardiac or skeletal muscle cells.

14. The method according to claim 12, wherein said cells are smooth muscle cells.

15. A method of treating endothelial dysfunction in a subject, said method comprising administering to the subject the nucleic acid delivery vehicle of any one of claims 1-7 or practicing the method according to any one of claims 8-14 on the subject.

16. A cell for the production of the nucleic acid delivery vehicle of any one of claims 1 to 7, said cell comprising means for producing virus vector in the absence of replication competent adenovirus and adeno-associated virus.

17. The cell of claim 16, wherein said cell expresses at least one means for the production of said virus vector from a nucleic acid integrated in the chromosomal DNA of said cell and expresses other means for producing of said virus vector from nucleic acid not integrated in the chromosomal DNA of said cell and wherein said integrated nucleic acid and said non-integrated nucleic acid do not comprise sequence overlap leading to the formation of replication competent adenovirus.

18. The cell of claim 17, wherein said integrated nucleic acid comprises at least an adenovirus E1-region or a functional analogue or a derivative thereof.

19. The cell of claim 17 or claim 18, wherein said integrated nucleic acid comprises at least a sequence encoding an adenovirus E2A protein, preferably an E2A-protein derived from adenovirus ts125 or functional analogues or derivatives thereof.

20. The cell of any one of claims 17-19, wherein said integrated nucleic acid comprises at least an adenovirus E4-region, preferably E4-orf6, or a functional analogue or a derivative thereof.

21. The cell of any one of claims 17-20, wherein said cell is derived from a PER.C6 cell (ECACC deposit number 96022940).
